(19) United States
(12) Patent Application Publication
(10) Pub. No.: US 2009/0292204 A1
(43) Pub. Date: Nov. 26, 2009

(54) METHOD AND DEVICE FOR RECOGNIZING TISSUE STRUCTURE USING DOPPLER EFFECT

(75) Inventor: Amir Pansky, Atlit (IL)

Correspondence Address: CADWALADER, WICKERSHAM & TAFT LLP, ONE WORLD FINANCIAL CENTER, NEW YORK, NY, 10281, US

(73) Assignee: OSCILLON LTD., Tel Aviv (IL)

(21) Appl. No.: 12/333,956

(22) Filed: Dec. 12, 2008

Related U.S. Application Data
(60) Provisional application No. 61/055,837, filed on May 23, 2008.

(51) Int. Cl. A61N 7/00; A61B 8/00

(52) U.S. Cl. 600/439

(57) ABSTRACT

An apparatus, method and system comprising an imaging tool for imaging an interventional device and its target organ using Doppler ultrasound are disclosed. The apparatus comprises an imaging tool and an interventional medical device; in some embodiments the imaging tool itself may be an interventional device or may comprise components of the interventional device. The imaging tool comprises a vibratory transducer and a vibratable element. The vibratory transducer is energized to generate a vibration motion in the vibratable element at first imaging frequency, which in turn is capable of oscillating a target organ at a second imaging frequency, the imaging frequencies being detectable by an ultrasound imaging system with Doppler mode. In certain applications the vibratory transducer may be energized to vibrate at a therapeutic frequency to effect treatment of the target organ, for example to clear a lumenal or vascular stenosis or to penetrate a vascular occlusion.

The present invention relates to ultrasonic imaging of interventional devices and target organs and, in particular, an imaging tool, comprising a vibratory transducer and a vibratable element, coupled to an ultrasonic imaging system that uses Doppler ultrasound. The vibratable element of the imaging tool is capable of rendering the interventional medical device and the target organ or internal tissue to be treated detectable to the imaging system thereby enhancing the visibility of each. The invention is particularly useful for interventional radiology and cardiology.

### BACKGROUND OF THE INVENTION

In order to treat organs in a minimally invasive manner, physicians use various imaging methods, both for imaging the treated organs and to determine the absolute and relative position of the invasive tool with respect to the targeted organ. Some common methods, for example, are: X-ray fluoroscopy for imaging endovascular catheterization treatment; computed tomography (CT) or magnetic resonance imaging (MRI) for interventional or diagnostic purposes, such as used by radiologists during tumor ablation procedures involving radiofrequency, cryosurgery, etc.; percutaneous ultrasound for imaging abdominal organs, such as kidney, liver and uterus; and laparoscopic ultrasound for surgical or diagnostic laparoscopy.

X-ray fluoroscopy is a technique widely used in endovascular procedures. Because adequate imaging requires injection of a contrast material into the vessel and adequate blood flow, however, its use is limited where blood flow is reduced or blocked. For example, if an artery is totally occluded—as in the case of chronic total occlusions (“CTO”)—the consequent absence of contrast material does not permit visualization of the designated artery and plaque to be treated. Therefore, the treating physician cannot use the technique to direct the invasive tool, wire, catheter or other dedicated device through the occlusion. CT and MRI provide high fidelity images and are minimally invasive, but due to high cost and the design of the machinery, these techniques generally are available for a limited number and type of interventional settings. Ultrasound, on the other hand, is widely used, but it provides relatively poorer image quality and requires a highly skilled operator to recognize structures and interpret the images, especially where complicated organs are at issue.

Ultrasonic imaging allows physicians to visualize organs and target internal tissues in a minimally invasive manner. Ultrasound waves directed toward target tissues are partially reflected off the internal organs and tissue structures they encounter. The reflected sound waves are processed by an ultrasonic receiver to produce an image viewable by the physician. Specifically, information—such as the relative time for reflected waves to reach the receiver and the strength of those waves—provide information regarding the location, size and density of the internal organs and tissue structures, thereby permitting physicians to view the shape and size of organs or locate lesions and potential tumors.

Ultrasound devices generate and detect sound waves; the behavior of sound waves makes them a useful tool in many fields, including medical imaging, radar in meteorology to track or evaluate storms, and in astronomy to measure the direction and speed of moving stars, defense, law enforcement, etc., because of the so-called Doppler effect. The Doppler effect describes the shift in frequency of sound (or light) waves reflected from a moving body—increasing if the body is moving toward a detection point or decreasing if the body is moving away. Thus, using an ultrasound transducer one can determine the location, direction and speed of the reflected object by measuring and calculating the change of sound wave frequency.

Doppler sonography is a preferred ultrasonic technique for medical imaging, because it permits visualization of moving objects relative to a static background. To treat a target organ or tissue using an interventional medical device, a physician must be able to aim the device accurately relative to the target; this requires the physician to be able to identify the location and position of the interventional medical device relative to the target organ or tissue or the portion of the organ or tissue to be treated. Measurement of Doppler shifts permits separation of moving objects from the background, such as movement of blood cells or the beating of the heart (as in an echocardiogram). Often, the moving objects are assigned a color depending on the direction of their motion and the static background is depicted in gray-scale. Several uses of ultrasound and Doppler in the medical art are described below.

There is a need in the art for an apparatus or system that permits Doppler imaging of both the interventional device and its target organ that is compatible with the therapeutic aspect of the interventional device.

Accordingly, it is an object of the invention to provide an imaging tool for use with an ultrasound system so as to permit imaging by Doppler ultrasound of both an interventional device and a target organ during a interventional procedure. It also is an object of the invention to provide a method of rendering an interventional device and target organ detectable and to provide a system comprising the apparatus and an imaging system.

### SUMMARY OF THE INVENTION

The apparatus, method and system of the invention provide a deliberate means for slightly oscillating a target organ using a dedicated imaging tool comprising a vibratable element, which enables the target organ to be imaged using Doppler ultrasound. As both the vibratable element and the target organ are moving, both can be imaged simultaneously as can their relative position and orientation. Such an imaging tool can incorporate or be deployed in standard interventional medical devices for treating the target organ.

In particular, the apparatus and method of the invention provide an imaging tool for use with an imaging system that enables an operator to visualize an interventional device and a target organ. In broad terms, the imaging tool comprises a vibratory transducer and a vibratable element. When energized, the vibratory transducer is capable of generating at least one vibration frequency for causing a vibration motion in the vibratable element, and the vibratable element is thereby capable of oscillating a target or target organ (the terms target and target organ are used interchangeably herein) with an amplitude, in particular with a small amplitude. The imaging tool is preferably used with an ultrasonic imaging system. The system of the invention comprises the imaging tool, the interventional device and the ultrasonic imaging system. The (in particular small amplitude, low frequency) oscillation of the target organ allows the image of the target organ to be emphasized over other tissues, using standard ultrasound or Doppler modes. The method of the invention preferably makes use of the ultrasonic imaging system to assist an operator in visualizing the target organ and the end of the interventional device responsible for treating the target organ.

The imaging tool may be placed in an interventional device, may itself be an interventional device, or it may employ components of the interventional device with which it is used. The vibratory transducer of the imaging tool of the invention is capable of vibrating the vibratable element at an imaging frequency. In particular, the vibratory transducer can generate a vibration motion in the vibratable element wherein the vibration motion has an imaging frequency preferably generating a speed in a Doppler detectable range—a “low frequency.” The vibratable element is preferably located at the distal end of the interventional device, i.e., the end of the interventional device designed to be closest to or in contact with a target organ. Such a vibratable element may also be referred to as a vibratory tip, and in particular applications—a therapeutic tip. When the vibratable element is made to vibrate at an imaging frequency and made to contact a target organ, the vibratable element is capable of causing the target organ to oscillate at an imaging frequency. Alternatively, the vibratable element may be designed to transfer the vibration motion to the target organ through an intermediate element and thereby cause the target organ to oscillate at an imaging frequency. The intermediate element may comprise or be any compatible material capable of transmitting the vibration motion to the target organ.

The apparatus of the invention, when not itself an interventional medical device, may be used with standard interventional medical devices. For example, the apparatus may be used with an endovascular catheter, on a guide wire or other probe used for re-vascularization of arteries or other vascular therapeutic purposes, or the apparatus may be used with an endolumenal catheter, trocar or other instrument used for non-vascular diagnostic or therapeutic purposes, for example a laparoscopic device. The vibratable element may be integral to the imaging tool or may be integral to the interventional device. Preferably the vibratable element is positioned so as to correspond to the distal end of the interventional device, and therefore also may be referred to as a vibratable tip. The vibratory transducer causes the vibratable element, or tip, to vibrate at a first imaging frequency, and the vibratable element, or tip, in turn causes the target organ, such as a vessel occlusion or plaque or an organ to be biopsied, to oscillate at a second imaging frequency, said first and second imaging frequencies being within a frequency range that produces a vibration detectable via Doppler shift. When the apparatus of the invention comprises an intravascular catheter, the apparatus or catheter may further comprise intravascular ultra sound (IVUS).

In some embodiments, in particular when the interventional device is an endovascular device, the vibratory transducer may be modulated to generate a second, in particular therapeutic frequency vibration in the vibratable element, in addition to the first imaging, frequency vibration, which therapeutic frequency is a higher frequency than the first imaging frequency, and is also referred to herein as a “high frequency.” The high frequency vibration of the vibratable element may be sufficient to effect, for example, penetration or traversal of a blood vessel occlusion. The vibratable element preferably is a vibratable tip, in particular a therapeutic tip, that can be integral to the interventional device, that may operate to penetrate and open the vessel or to clear the vessel when made to vibrate at the second, in particular a high or therapeutic, frequency and made to contact a vessel occlusion or vascular stenoses.

The invention also provides a system for imaging a therapeutic device and a target organ, wherein the system comprises the apparatus of the invention and an ultrasound imaging system with Doppler mode. The invention further provides a method of rendering an interventional device and target organ detectable to an operator using the apparatus or system of the invention. The invention still further provides a method of rendering an interventional device and target organ detectable and also effecting an interventional outcome on said target organ.

In any of the embodiments, the whole apparatus may be disposable or the imaging tool may be used with a disposable interventional medical device.

### BRIEF DESCRIPTION OF DRAWINGS

FIG. 1 is a schematic representation of an embodiment of the imaging tool according to the invention, depicting the principle of operation.

FIG. 2 is a schematic representation of an embodiment of the invention where the imaging tool is used with an IVUS for lesion visualization with or without an ultrasound imaging system.

FIG. 3 is a schematic representation of an embodiment of the invention where the imaging tool includes a vibratory transducer that is capable of being modulated between a low frequency for imaging and a higher frequency as necessary for penetrating a blood vessel occlusion.

FIG. 4 is a schematic representation of an embodiment of the invention where the imaging tool is used to visualize a laparoscopic device comprising a biopsy tip.

### DETAILED DESCRIPTION OF THE INVENTION

The present invention is directed to an apparatus, method and system for visualizing the distal end of an interventional device and the organ or lesion that is the target of the interventional device. The apparatus of the invention comprises an imaging tool for use with an ultrasound imaging apparatus. The imaging tool comprises a vibratory transducer and a vibratable element. The vibratory transducer is capable of generating vibrational energy to effect a vibration motion in the vibratable element at first imaging frequency of about 2 Hz to about 2 kHz (a low frequency), hereinafter also referred to as simply a first frequency. The vibratable element is capable of causing a target organ to oscillate at a second imaging frequency of about 2 Hz to about 2 kHz (a low frequency), with an amplitude of less than about 2 mm peak to peak (hereinafter referred to as a small amplitude), such that the operator can introduce the imaging tool into a body tissue or lumen in need of treatment and visualize both the vibratable element and the target organ, or relevant portion thereof, and thereby be assisted in treating the target organ or effecting a desired interventional result on said target or target organ. The first imaging frequency of the vibratable element and second imaging frequency of the target may be the same frequency or different frequencies, but both are within the “low frequency” range of about 2 Hz to about 2 kHz. Preferably, the low frequency vibration provides a vibration speed of about 1-200 cm/s for Doppler detection.

The apparatus of the invention may be deployed within an interventional medical device or itself may comprise an interventional medical device. In one embodiment, the apparatus is an imaging tool comprising a vibratory transducer functionally connected to a vibratable element that is deployable in an interventional device; in another embodiment, the apparatus of the invention is the imaging tool comprising a guide wire; in still another embodiment, the apparatus of the invention is an endovascular catheter, comprising the imaging tool.

For example, in one embodiment, the apparatus of the invention may be guided to the target organ within an interventional medical device, such as a catheter, optionally in conjunction with a guide wire, within a trocar, with other interventional devices known to those in the art, or independently. The apparatus may be introduced into a blood vessel and guided to the target organ within an endovascular catheter having a guide wire. Such an endovascular catheter may further comprise IVUS for guiding the catheter to the target organ or lesion. In another embodiment, for example, the apparatus itself is an interventional medical device, comprising the components of the imaging tool. In either embodiment, the vibratable element of the imaging tool may be positioned at, e.g., the distal end of an interventional device, so that the visualized position of vibratable element represents, e.g., the position of the distal end of the interventional device, where the therapeutic, diagnostic or other interventional activity occurs. The imaging tool may be disposable or may be used in a disposable interventional device.

More particularly, the apparatus of the invention comprises an imaging tool, which imaging tool comprises a vibratory transducer, for example a vibration motor, and a vibratable element. The vibratory transducer and vibratable element can be capable of cooperatively causing a target organ to oscillate at a second imaging frequency (a low frequency) to enhance the detectability of the target organ and the distal end or therapeutic tip of a interventional device, whether imaged using standard ultrasound or Doppler modes. The apparatus of the invention preferably is used with an imaging system having Doppler imaging capability that permit an operator, such as a physician, to image both the therapeutic end of the interventional device and the target organ to be treated, using Doppler ultrasound, to achieve an minimally invasive yet accurate placement of the interventional device. The movement of both the vibratable element and the target organ may be detected and processed by an imaging system with Doppler mode into information useful to the operator for guiding the interventional device relative to the target organ during a therapeutic or diagnostic procedure.

The imaging system preferably is an ultrasonic imaging system, comprising an ultrasonic transducer and a processor, which ultrasonic transducer is capable of sending ultrasound energy waves and receiving return wave signals reflected off of the vibratable element, the target organ and background tissues, and measuring motion using the Doppler effect. The imaging system may comprise receivers for receiving reflected signals. The processor may process the reflected signals and measure the movements of the vibratable element and target organ based on the Doppler effect. The processor further is capable of generating useful information from the signals and measurements, to assist an operator in using the interventional device. In particular, the processor may be capable of generating images useful for visualizing the location of the vibratable element and the target organ relative to the surrounding environment. The processor may be, for example, a laptop or desktop computer. When the vibratable element is positioned at the distal end of the interventional device, the location of the vibratable element represents the location of the distal end of the interventional device. Preferably the ultrasonic imaging system further comprises an image screen for displaying the images. The ultrasonic transducer may be, for example, an abdominal, laparoscopic, transrectral, vaginal, or intravascular transducer or any other applicable ultrasound transducer known in the art. In one embodiment, the ultrasound imaging system further comprises IVUS.

In particular embodiments, the apparatus of the invention is designed to enable the opening of a vessel occlusion or the clearing of a vascular plaque. In such embodiments, the interventional device is an endovascular catheter and the vibratory transducer of the imaging tool is capable of providing a plurality of vibration frequencies and may be modulated, for example, to generate a first frequency vibration, in particular a low frequency vibration, for Doppler imaging and a second frequency vibration of about 2 kHz to about 1 MHz (hereinafter also referred to as a therapeutic frequency or a high frequency vibration) for therapeutic purposes. The vibratory transducer is capable of causing a vibratable element, e.g., a vibratable tip, more particularly a vibratable therapeutic tip, to vibrate at said plurality of vibration frequencies, including said second, or therapeutic, frequency, which therapeutic frequency vibration may be sufficient to effect penetration and traversal of a vessel occlusion and/or clearing plaques, or stenoses, from a vessel. The vibratable element, or vibratable tip, preferably is integral to the interventional device, such as the distal tip of a guide wire, a vibration cap at the end of an endovascular catheter, or an expandable or elastic membrane.

For example, in one embodiment, the apparatus of the invention is capable of being delivered into a blood vessel, and the imaging tool comprises a vibratory transducer that generates and transmits a plurality of vibration frequencies to a vibratable element, preferably located at the distal end of an endovascular catheter. The plurality of vibration frequencies includes a first frequency, in particular a first imaging frequency, to vibrate the vibratable element which in turn vibrates a target organ at a second imaging frequency with small amplitude (e.g., <2 mm peak to peak) so as to permit imaging of both the interventional device and the target organ by Doppler ultrasound during the therapeutic interventional procedure. The plurality of vibration frequencies also includes a second frequency, in particular a therapeutic frequency, to vibrate the vibratable element at a frequency sufficient to enable penetration and traversal of a blood vessel occlusion, the vibratable element being configured as a therapeutic tip for penetration of blood vessel occlusion. In such embodiments, when the vibratable tip (or therapeutic tip) is made to encounter a vessel occlusion or vascular stenoses and is made to vibrate at said second frequency, in particular high frequency, it may operate to penetrate and open the vessel or to clear the vessel, for example by pneumatic drilling and/or cavitation, depending on the therapeutic vibration frequency and material encountered. In another embodiment, the apparatus of the invention is capable of being delivered into a body lumen having a stenosis, and in a similar manner may be used to visualize the interventional device and the stenosis and to penetrate and clear the lumenal stenosis.

In one embodiment of the invention, the vibratory transducer of the imaging tool is a piezoelectric motor. In a preferred embodiment the piezoelectric motor is a piezoelectric crystal or a piezoelectric ceramic. The piezoelectric motor may be positioned at the distal end of the imaging tool, or alternatively the piezoelectric motor may be positioned at the proximal end of the imaging tool.

The invention also provides a method of rendering an interventional device and target organ detectable to an operator using any one of the apparatuses of the invention. The apparatus of the invention, comprising a vibratory transducer and a vibratable element located at the distal end of an interventional device, is introduced into the body of a patient in need of treatment. The vibratory transducer, which is capable generating plurality of frequencies, is made to cause the vibratable element to vibrate at a first imaging frequency—a low frequency—to enhance the detectability of the vibratable element. The vibratable element is made to contact a target organ and thereby cause the target organ to oscillate at a second imaging frequency—a low frequency—to enhance the detectability of the target organ. An ultrasound transducer is provided, which is capable of sending ultrasound waves and detecting the returned (reflected) ultrasound wave signals. The ultrasound transducer is part of an imaging system, which is used to detect the reflected signals, as well as movements of the vibratable element and target organ, measured using the Doppler shift. The information is used to reveal the location of the vibratable element and target organ relative to each other and the surrounding environment. Images are generated on an image screen, which may be used to guide the interventional device during a therapeutic or diagnostic procedure.

Also provided is a method of rendering an interventional device and target organ detectable and also effecting interventional outcome on said target organ, comprising introducing into the environs of the target organ an interventional device comprising an imaging tool, said imaging tool comprising a vibratory transducer and vibratable element located at the distal end of said interventional device, said vibratory transducer being capable of vibrating said vibratable element at a plurality of vibration frequencies, including a first frequency (low frequency) imaging mode to vibrate the organ at small amplitude and a second frequency (high frequency) effector mode sufficient to enable interventional effect; energizing said vibratory transducer to generate a small amplitude vibration and causing said vibratable element by direct or indirect contact to vibrate the target organ; providing an ultrasound system having a Doppler detection mode; generating images of both the vibratable element and the target organ with the ultrasound system in Doppler mode to guide the interventional device relative to the target organ; modulating the vibratory transducer to generate an effector mode frequency; and effecting intervention on the target organ using the vibratable element as an effector tip.

In a particular embodiment of this method, a method of rendering an interventional catheter and blood vessel occlusion or plaque detectable and also treating said blood vessel occlusion or plaque is provided. An interventional catheter comprising the apparatus of the invention is introduced into a blood vessel having an occlusion or stenoses, the apparatus comprises a vibratory transducer capable of vibrating a vibratable element at a plurality of vibration frequencies, including a low frequency imaging mode sufficient to vibrate an occlusion or plaque at a small amplitude and a high frequency therapeutic mode sufficient to enable penetration and/or cavitation of the occlusion or plaque. The vibratory transducer is energized to generate a small amplitude vibration and cause the vibratable element, by direct or indirect contact, to vibrate the occlusion or plaque. Images of both the vibratable element and the vibrating occlusion or plaque may be generated using an ultrasound system having a Doppler detection mode, and used to guide the interventional catheter during the therapeutic process. The vibratory transducer may be modulated to generate a therapeutic mode frequency so that the vibratable element may be used as a therapeutic tip to penetrate and/or cavitate the occlusion or plaque.

The invention still further provides a system for imaging a therapeutic device and a target organ, comprising an interventional medical device, an imaging tool comprising a vibratable element and a vibratory transducer for vibrating the vibratable element at a low frequency, wherein the vibratable element is positioned to mark the distal end of the interventional medical device and is capable of causing a target organ to vibrate at that low frequency, and an ultrasound imaging system comprising Doppler detection receivers for detecting the movement of the vibratable element and target organ and a processing unit for providing an image of the relative position of the distal end of the interventional medical device and the target organ based on signals from the receivers.

The following detailed description should be read with reference to the drawings, in which similar elements in different drawings have numbers with identical last two digits. The drawings, which are not necessarily to scale, depict selected embodiments and are not intended to limit the scope of the invention as set forth in the claims.

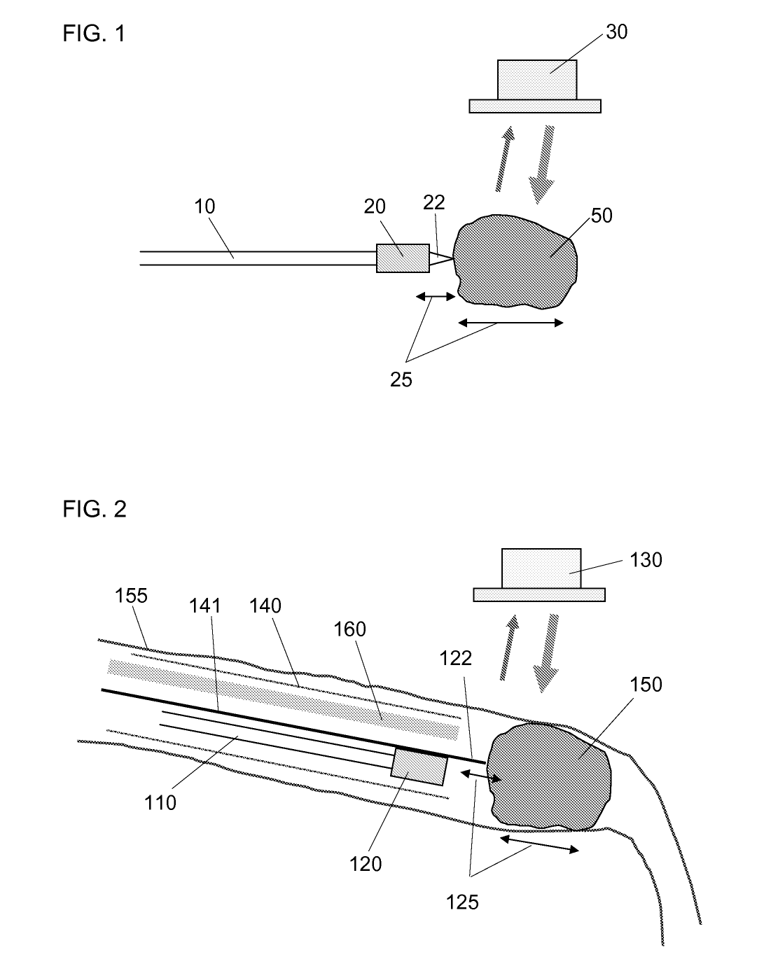

The principle of operation of the apparatus of the invention is schematically depicted in FIG. 1. The imaging tool 10 comprises a vibratory transducer 20, in this case a vibration motor, and a vibratable element 22. The vibratory transducer 20 is capable of generating at least one vibration frequency, including a first imaging frequency of about 2 Hz to about 2 kHz, and transmitting the first imaging frequency to the vibratable element 22. The vibratable element 22 is capable of vibrating at the first imaging frequency, and transmitting vibration to the target organ 50 to vibrate the target organ 50 at a second imaging frequency of about 2 Hz to about 2 kHz. The movement 25 of both the vibratable element 22 and the target organ 50 are detectable by an ultrasonic transducer 30, which may be, for example, an ultrasound laparoscopic transducer, an ultrasound abdominal transducer, an ultrasound transrectal transducer, or an ultrasound vaginal transducer. The detected signals are transmitted to an imaging system (not shown) where the motion and relative positions of the vibratable element 22 and target organ 50 are displayed for use by the operator. The imaging tool 10 depicted in FIG. 1 may be deployed in an interventional device (not shown), however in alternative embodiments the imaging tool may itself be an interventional device or the vibratable element may comprise a component of an interventional device.

FIG. 2 depicts an embodiment of the apparatus of the invention in situ in a blood vessel 155 having an occlusion, which in this embodiment is the “target organ” 150. The imaging tool 110 is introduced into the blood vessel 155 within an interventional device, in particular an endovascular catheter 140, comprising a plurality of lumens (not shown), which lumens hold the imaging tool 110, a guide wire 141, and optionally an IVUS device 160. The imaging tool 110 comprises a vibratory transducer 120 (in this case a vibration motor) capable of vibrating a vibratable element 122, in this case the distal end of the guide wire 141, at a first frequency, in particular a first imaging frequency, of about 2 Hz to about 2 kHz. FIG. 2 depicts the vibratable element 122 transmitting a vibration to the target organ 150, in this case a vessel occlusion, to oscillate the vessel occlusion at a second imaging frequency of about 2 Hz to about 2 kHz. The movement 125 of both the vibratable element 122 and the target organ 150 (occlusion) are detectable by an ultrasonic transducer 130, for example, an ultrasound abdominal transducer or an ultrasound thoracic transducer. The detected signals are transmitted to an imaging system (not shown) where the motion and relative locations of the vibratable element 122 and target organ 150 (occlusion) relative to surrounding environment, such as the wall of the blood vessel 155, are displayed for use by the operator. In this embodiment, the imaging system (not shown) may also process information from the IVUS 160.

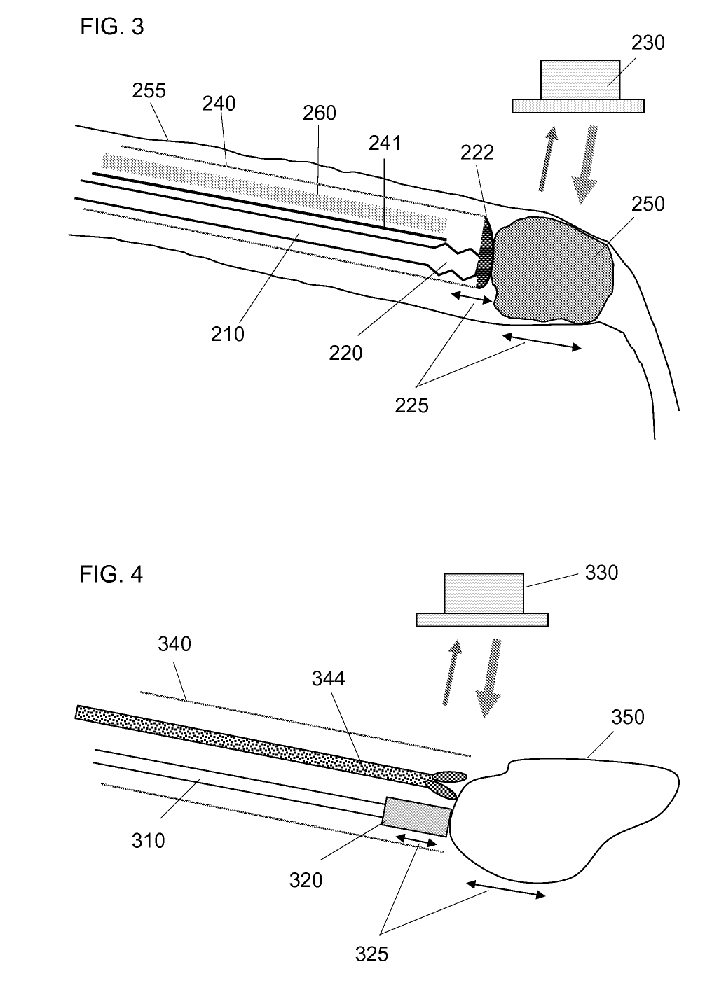

FIG. 3 depicts an embodiment of the apparatus of the invention in situ in a blood vessel 255 having an occlusion, which in this embodiment is the “target organ” 250. The apparatus of this embodiment comprises an interventional device 240, in particular an endovascular catheter that further comprises a guide wire 241 and optionally an IVUS device 260, and an imaging tool 210 comprising a vibratory transducer 220 and vibratable element 222, in this case a vibratable tip, or therapeutic tip, more particularly a vibration cap, at the distal end of the interventional device 240. In this embodiment, the imaging tool 210 lies within the interventional device 240 and thereby may be introduced into the blood vessel 255. The vibratory transducer 220 of this embodiment may be modulated to operate at a plurality of frequencies—a first (low) frequency of about 2 Hz to about 2 kHz for imaging and a second (high) frequency of about 10 kHz to about 40 MHz for therapeutic purposes. The imaging tool 210 may be operated at both frequencies, or it may be operated only at the first (imaging) frequency. When the vibratory transducer 220 is made to vibrate the vibratable element 222 at a low frequency, and the vibratable element 222 is made to come in contact with the target organ, the vibratable element 222 is capable of causing an imaging frequency (low frequency) vibration in the target organ 250, in this case a vessel occlusion, to oscillate the occlusion with small amplitude (<2 mm peak to peak). The movement 225 of both the vibratable element 222 and the target organ 250 (occlusion) may be detected and the Doppler shift may be measured by an ultrasonic transducer 230 with Doppler capability. The ultrasonic transducer may be, for example, an ultrasound abdominal transducer, an ultrasound thoracic transducer, an ultrasound laparoscopic transducer, or other appropriate ultrasound transducer capable of operating in Doppler mode. The detected signals and measurements may be transmitted to an imaging system (not shown) where the motion and relative positions of the vibratable element 222 and the target organ 250 may be displayed for use by an operator. In this embodiment, the imaging system (not shown) may also process the IVUS 260 information.

The vibratory transducer 220 of the embodiment depicted in FIG. 3 may be modulated to vibrate the vibratable element 222 at a second (high) frequency. At this high frequency, the vibratable element 222 is capable of functioning in a therapeutic mode to penetrate or clear the target organ 250, in this case the vessel occlusion or plaque material (stenosis). The embodiment is designed to allow the operator to modulate the vibration frequency of the vibratory transducer 220 between the first (imaging) frequencies and the second (therapeutic) frequencies as needed during a treatment procedure. The vibratory transducer 220 may be a vibration motor, such as a piezoelectric motor, as shown in FIG. 3, or other electromechanical transducer known in the art. Alternatively, the transducer may comprise a hydraulic-based vibrational energy source and hydraulic lumen, as described by way of example in FIGS. 1, 2, 4, 5 and 6 of co-pending application Ser. No. 12/325,868, filed Dec. 1, 2008, entitled “Method And Device For Recanalization Of A Total Occlusion”, which is incorporated herein by reference in its entirety.

Briefly, in embodiments where the vibratory transducer is based on a hydraulic energy, it comprises an external hydraulic energy source to generate hydraulic wave pulses into a hydraulic lumen of a hydraulic catheter. The hydraulic lumen is designed to transmit the hydraulic wave pulses to a vibratable element. The vibratable element in such an embodiment would be comparable to the vibrating member of the above-referenced co-pending application Ser. No. 12/325,868, as shown by way of example in FIGS. 2, 4, 5 and 6 of that co-pending application. As also described in the above-referenced co-pending application Ser. No. 12/325,868, a system comprising the hydraulic energy-based vibratory transducer additionally includes a control unit for adjusting the vibration frequency and/or vibration amplitude, thus as applicable in the present invention such a system may be used to modulate vibration of the vibratable element, at first frequencies, in particular imaging frequencies, such that the vibration motion is detectable by Doppler ultrasound and at second frequencies, in particular therapeutic frequencies, sufficient to permit the vibratable element to penetrate an occlusion or clear a plaque.

The vibratable element may comprise, for example, a guide wire, a bellows, an elastic membrane or a vibration cap. Examples of bellows and elastic membranes as vibratable elements in a hydraulic-based system are described, for example, in the above-referenced co-pending application Ser. No. 12/325,868, which is incorporated herein by reference, in particular as described for FIGS. 2, 4 and 5 therein. Examples of guide wires as vibratable elements in a hydraulic-based system are described, for example, in the above-referenced co-pending application Ser. No. 12/325,868, in particular as described for FIG. 6 therein, and in a piezoelectric motor-based system are described in co-pending U.S. application Ser. No. 12/126,665, filed May 23, 2008, in particular as described for FIGS. 2 and 3, therein.

In other embodiments of the invention, the imaging tool may be used in a lithotripsic device, or in a laparoscopic device as schematically depicted in FIG. 4. In particular, the imaging tool 310 of the apparatus of this embodiment comprises a vibratory transducer 320 and vibratable element 322, deployed in an interventional device 340, for example within a laparoscopic-type instrument as shown in FIG. 4. In this embodiment, the imaging tool 310 may be used in lieu of a fiber optic camera in the laparoscopic device. The imaging tool 310 is introduced into a body cavity within the interventional device 340, which comprises a plurality of lumens (not shown), which lumens hold a diagnostic or surgical device, in this case a biopsy tool 344, and components supporting the vibratory transducer 320. The vibratory transducer 320 of this embodiment can be made to vibrate the vibratable element 322 at an imaging (low) frequency. The vibratable element 322 transmits a vibration to the target organ 350, causing the target organ 350 to oscillate at an imaging (low) frequency. The movements 325 of both the vibratable element 322 and the target organ 350 may be detected via reflected ultrasonic signals, and the Doppler shift may be measured, by an ultrasonic transducer 330. The ultrasonic transducer may be, for example, an ultrasound laparoscopic transducer, an ultrasound abdominal transducer, an ultrasound transvaginal transducer or any other appropriate ultrasound transducer capable of operating in Doppler mode. The detected signals and Doppler shift measurements may be transmitted to an imaging system (not shown) where the motion and relative positions of the vibratable element 322 and the target organ 350 may be displayed for use by an operator. In an alternative embodiment, the imaging tool 310 may be deployed in a catheter separate from the laparoscopic device.

Optionally, where actual measurement of the vibration speed is desired, the imaging tool may further comprise a sensor. Sensors that may be useful for such purposes include those described in detail in the above-referenced co-pending application Ser. No. TBA, for example, a strain gauge sensor, a piezoresistor, or a microstrain sensor, which may be adapted for measuring the speed of vibration of either or both of the vibratable element and target. For example, a sensor located at the distal end of the imaging tool may provide feedback to a processor that generates a readable output for the operator who can, via the control unit, manually adjust the energy input from the energy source to the vibratory transducer. Alternatively, the processor in conjunction with the control unit may be designed to automatically adjust the energy input to the vibratory transducer based on information received from such a sensor to ensure a vibration speed of 1-200 cm/s.

The invention further encompasses methods of using any one of the embodiments of the apparatus of the invention. In one embodiment, for example using the imaging tool depicted in FIGS. 2 or 3, a method of rendering an interventional device and a target organ detectable to an operator is provided. The invention also provides a method of rendering an interventional medical device and a target organ detectable. In one embodiment, the method comprises (a) introducing into a patient's body an apparatus comprising (i) an interventional medical device having a proximal end and a distal end and (ii) an imaging tool, said imaging tool comprising a vibratory transducer, and a vibratable element, wherein said vibratory transducer is capable of vibrating said vibratable element at a first imaging frequency of about 2 Hz to about 2 kHz, and wherein said vibratable element is located at said distal end of said interventional medical device and is capable causing a target organ to oscillate at a second imaging frequency of about 2 Hz to about 2 kHz; (b) advancing said distal end of said interventional medical device toward said target organ; (c) causing said vibratory transducer to vibrate said vibratable element at said first imaging frequency; (d) causing said vibratable element to oscillate said target organ at said second imaging frequency; (e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected; (f) employing said ultrasound imaging system to receive said frequency signals, measure and process said Doppler shifts, and generate and display said images; and (g) using said images to visualize both said target organ and said distal end of said interventional medical device during an interventional procedure. In an alternative embodiment of this method, the imaging tool is the interventional device introduced into the patient's body. The interventional device may be an endovascular catheter comprising a guide wire, as depicted in FIG. 2, an endolumenal catheter, a laparoscopic device or a trocar. The target organ may be any organ, substance or material in the body in need of treatment or an interventional procedure. The vibratory transducer may be any of the vibratory transducers, as described above.

Another embodiment is a method of rendering an interventional device and a target organ detectable to an operator and treating said target organ, as shown for example in FIG. 3. In this embodiment, the method comprises using the apparatus in an imaging mode to vibrate a target organ, so as to permit detection of both the interventional device and the target organ, and modulating the vibration frequency to a therapeutic mode, wherein the vibratable element, which may be a vibratable tip or therapeutic tip, is made to vibrate at a therapeutic frequency, e.g., a frequency sufficient to penetrate an occlusion or ablate a plaque in a blood vessel. Specifically, in one embodiment, the method comprises (a) introducing into a blood vessel having an occlusion or stenosis an apparatus comprising (i) an interventional medical device having a proximal end and a distal end and (ii) an imaging tool, said imaging tool comprising a vibratory transducer and a vibratable element, wherein said vibratable element is located at said distal end of said interventional medical device such that it is capable of contacting said vessel occlusion or stenosis, and wherein said vibratory transducer is capable of generating a plurality of vibration frequencies for vibrating said vibratable element at a first imaging frequency of about 2 Hz to about 2 kHz for Doppler imaging and at a second frequency of about 10 kHz to about 1 MHz sufficient for causing penetration or cavitation of vessel occlusions or clearing vessel stenosis; (b) advancing said distal end of said interventional medical device toward a proximal surface of said occlusion or stenosis; (c) causing said vibratory transducer to vibrate said vibratable element at said first imaging frequency; (d) advancing said interventional device until said vibratable element contacts said occlusion or stenosis, such that said vibratable element transmits a vibration to said occlusion or stenosis, causing said occlusion or stenosis to oscillate at said second imaging frequency; (e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected; (f) employing said ultrasound imaging system to receive said frequency signals, measure and process said Doppler shifts, and generate and display said images; (g) modulating said vibration frequency of said vibratory transducer to said high frequency and causing penetration or cavitation of said occlusion or at least partial ablation of said stenosis; and (h) alternately (i) locating a new proximal surface of said occlusion or stenosis and (ii) penetrating said occlusion or ablating said stenosis by modulating said vibration frequency of said vibratory transducer between said low frequency and high frequency, until said occlusion is re-canalized or said stenosis is cleared. In this embodiment, the imaging tool provides a therapeutic component of the interventional device, as depicted in FIG. 3.

In another embodiment, the treating step of the method involves clearing a stenosis in a body lumen, such as the biliary tract or gastrointestinal tract, or any other body lumen known in the art to suffer stenoses. This method comprises (a) introducing into a body lumen having a stenosis an apparatus comprising (i) an interventional medical device having a proximal end and a distal end and (ii) an imaging tool, said imaging tool comprising a vibratory transducer and a vibratable element, wherein said vibratable element is located at said distal end of said interventional medical device such that it is capable of contacting said lumenal stenosis, and wherein said vibratory transducer is capable of generating a plurality of vibration frequencies for vibrating said vibratable element at a first frequency, in particular a first imaging frequency, of about 2 Hz to about 2 kHz for Doppler imaging and at a second frequency, in particular a therapeutic frequency, of about 10 kHz to about 1 MHz sufficient for clearing a lumenal stenosis; (b) advancing said distal end of said interventional medical device toward a proximal surface of said lumenal stenosis; (c) causing said vibratory transducer to vibrate said vibratable element at said first imaging frequency; (d) advancing said interventional device until said vibratable element contacts said lumenal stenosis, such that said vibratable element transmits a vibration to said lumenal stenosis, causing said lumenal stenosis to oscillate at said second imaging frequency; (e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected; (f) employing said ultrasound imaging system to receive said frequency signals, measure and process said Doppler shifts, and generate and display said images; (g) modulating said vibration frequency of said vibratory transducer to said therapeutic frequency and causing at least partial ablation of said lumenal stenosis; and (h) alternately (i) locating a new proximal surface of said lumenal stenosis and (ii) ablating said lumenal stenosis by modulating said vibration frequency of said vibratory transducer between said first imaging frequency and therapeutic frequency, until said lumenal stenosis is cleared.

In an alternative embodiment of the method of visualizing and treating a body organ, does not comprise a therapeutic mode vibration frequency. Specifically, the method comprises (a) introducing into a patient's body near a target organ an apparatus comprising (i) an interventional medical device having a proximal end and a distal end and (ii) an imaging tool, said imaging tool comprising a vibratory transducer and a vibratable element, wherein said vibratory transducer is capable of generating a vibration motion in said vibratable element, said vibration motion having a first imaging frequency of 2 Hz to about 2 kHz for Doppler imaging, wherein said vibratable element is located at said distal end of said interventional medical device and is capable of causing said target organ to oscillate at a second imaging frequency of 2 Hz to about 2 kHz for Doppler imaging, said target organ being in need of treatment, said interventional medical device being designed to treat said target organ; (b) advancing said distal end of said interventional medical device toward said target organ; (c) causing said vibratory transducer to vibrate said vibratable element at said first imaging frequency; (d) advancing said interventional medical device until said vibrating vibratable element contacts said target organ, and causing said target organ to oscillate at said second imaging frequency; (e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected; (f) employing said ultrasound imaging system to receive said frequency signals, measure and process said Doppler shifts, and generate and display said images; (g) using said images to visualize both said target organ and said distal end of said interventional medical device; and (h) treating said body organ with said interventional medical device. In one aspect of this embodiment, the interventional device may be an endovascular catheter with a therapeutic tip for ablating an occlusion, but the imaging tool is separate from the therapeutic component. The imaging tool functions to assist in accurate placement of the therapeutic component for safe and effective revascularization of the blood vessel. In another aspect of this embodiment, the interventional device may be a trocar or laparoscopic device adapted, for example, for sampling tissues for biopsy as depicted in FIG. 4. In such an embodiment, said target organ is an organ requiring a biopsy. In still another aspect of this embodiment, the interventional device may be an endolumenal catheter adapted, for example, for diagnostic use or exploratory surgery.

The invention further provides a system for imaging an interventional device and a target organ comprising the imaging tool or apparatus depicted in FIGS. 1-4. In one embodiment, the system of the invention comprises an interventional medical device having a proximal end and a distal end; an imaging tool comprising a vibratory transducer and a vibratable element, wherein said vibratory transducer is capable of generating at least one vibration frequency, including a first imaging frequency of about 2 Hz to about 2 kHz for generating a vibration motion in said vibratable element, and wherein said vibratable element is located at said distal end of said interventional medical device and is capable of causing a target organ to oscillate at a second imaging frequency of about 2 Hz to about 2 kHz with small amplitude; an energy source functionally connected to said vibratory transducer; a control unit functionally connected to said energy source, said control unit being capable of controlling and modulating said at least one vibration frequency generated by said vibratory transducer via said energy source; and an ultrasound imaging system for enabling an operator to localize said distal end of said interventional medical device and said target organ relative to surrounding tissues, said ultrasound imaging system comprising (i) an ultrasonic transducer for sending and receiving Doppler ultrasound wave signals, said ultrasonic transducer being capable of detecting Doppler shifts from movements of said vibrating vibratable element and said vibrating target organ, (ii) a processor and (iii) an image screen, wherein said ultrasonic transducer, said processor and said image screen are functionally connected. In another embodiment, the catheter may be an endovascular catheter that further comprises a guide wire and an IVUS component.

In a particular embodiment of the system of the invention, components for treating the target organ are provided. In one aspect of this particular embodiment in which the target organ may be a vascular plaque or a total occlusion in a blood vessel, the interventional medical device is an endovascular catheter, and the vibratory transducer is capable of providing a plurality of vibration frequencies, including a first frequency, in particular a first imaging frequency, comprising a low frequency vibration of about 2 Hz to about 2 kHz and a second frequency, in particular a therapeutic frequency, comprising a high frequency vibration of about 10 kHz to 1 MHz, and wherein said high frequency vibration is sufficient to enable said vibratable element to penetrate and traverse said vascular occlusion or to clear said vascular stenoses. The endovascular catheter may further comprise a guide wire for penetrating or clearing an occlusion or plaque in a blood vessel, which serves as the vibratable element of the imaging tool. In another aspect of this particular embodiment of the system of the invention, in which the target organ may be a lumenal stenosis, the interventional medical device is an endolumenal catheter, and the vibratory transducer is capable of providing a plurality of vibration frequencies, including a first frequency of about 2 Hz to about 2 kHz and a second frequency of about 10 kHz to 1 MHz, and said second frequency vibration is sufficient to enable said vibratable element to clear said lumenal stenosis.

It will be appreciated by persons having ordinary skill in the art that many variations, additions, modifications, and other applications may be made to what has been particularly shown and described herein by way of embodiments, without departing from the spirit or scope of the invention. Therefore it is intended that scope of the invention, as defined by the claims below, includes all foreseeable variations, additions, modifications or applications.

What is claimed is:

1. An apparatus for use with an ultrasound imaging system comprising:
an imaging tool comprising a vibratory transducer and a vibratable element, wherein said vibratory transducer is capable of generating at least one vibration frequency for causing a vibration motion in said vibratable element for oscillating a target organ with small amplitude.

2. The apparatus of claim 1, wherein said imaging tool is an interventional medical device having a proximal end and a distal end.

3. The apparatus of claim 1, wherein said apparatus further comprises an interventional medical device having a proximal end and a distal end.

4. The apparatus of claim 3, wherein said imaging tool is located within said interventional medical device.

5. The apparatus of claim 4, wherein said interventional medical device is an endovascular catheter and said imaging tool is an endovascular guide wire.

6. The apparatus of any one of claims 2-4, wherein said interventional medical device is an endovascular catheter.

7. The apparatus of claim 3, wherein said interventional medical device is an endolumenal catheter.

8. The apparatus of claim 3, wherein said interventional medical device is a laparoscopic device.

9. The apparatus of claim 3, wherein said interventional medical device is a trocar.

10. The apparatus of claim 2 or 3, wherein said vibratory transducer and said vibratable element are located at said distal end of said interventional medical device.

11. The apparatus of claim 2 or 3, wherein said vibratory transducer is located at said proximal end of said interventional medical device and said vibratable element is located at said distal end of said interventional medical device.

12. The apparatus of claim 3, wherein said vibratory transducer is external of said interventional medical device and said vibratable element is located at said distal end of said interventional medical device.

13. The apparatus of claim 1, wherein said vibratory transducer is capable of generating a plurality of vibration frequencies, including an imaging frequency of about 2 Hz to about 2 kHz and a therapeutic frequency of about 10 kHz to about 1 MHz.

14. The apparatus of claim 1, wherein said vibratory transducer is a piezoelectric motor.

15. The apparatus of claim 14, wherein said piezoelectric motor is a piezoelectric crystal.

16. The apparatus of claim 14, wherein said piezoelectric motor is a piezoelectric ceramic.

17. The apparatus of claim 1, wherein said vibratory transducer is an electro-mechanical transducer.

18. The apparatus of claim 1 or 12, wherein said vibratory transducer is a hydraulic energy source capable of generating hydraulic pressure waves or pulses.

19. The apparatus of claim 1, wherein the vibratable element is selected from the group consisting of: a guide wire, a bellows, an elastic membrane, and a vibrating cap.

20. The apparatus of claim 1, wherein said apparatus is functionally connected to an ultrasound system having Doppler imaging capability.

21. The apparatus of claim 20, wherein said ultrasound system comprises an ultrasound transducer selected from the group consisting of an intra-vascular transducer, a laparoscopic transducer, an abdominal transducer, a thoracic transducer, a transrectal transducer and a transvaginal transducer.

22. A method of rendering an interventional medical device and a target organ in a patient detectable comprising:
a) introducing into a patient's body an apparatus comprising (i) an interventional medical device having a proximal end and a distal end and (ii) an imaging tool, said imaging tool comprising a vibratory transducer and a vibratable element, wherein said vibratory transducer is capable of vibrating said vibratable element at a first imaging frequency of about 2 Hz to about 2 kHz, and wherein said vibratable element is located at said distal end of said interventional medical device and is capable causing a target organ to oscillate at a second imaging frequency of about 2 Hz to about 2 kHz;
b) advancing said distal end of said interventional medical device toward said target organ;
c) causing said vibratory transducer to vibrate said vibratable element at said first imaging frequency;
d) causing said vibratable element to oscillate said target organ at said second imaging frequency;
e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected;
f) employing said ultrasound imaging system to receive said frequency signals, measure and process said Doppler shifts, and generate and display said images; and
g) using said images to visualize both said target organ and said distal end of said interventional medical device during an interventional procedure.

23. A method of rendering an interventional medical device and a target organ in a patient detectable comprising:
a) introducing into a patient's body an imaging tool comprising a vibratory transducer and a vibratable element, wherein said imaging tool is an interventional medical device comprising a proximal end and a distal end, wherein said vibratory transducer is capable of generating a vibration motion in said vibratable element, said vibration motion having a first imaging frequency of about 2 Hz to about 2 kHz, and wherein said vibratable element is located at said distal end of said interventional medical device and is capable of causing a target organ to oscillate at a second imaging frequency of about 2 Hz to about 2 kHz;
b) advancing said distal end of said interventional medical device toward a target organ;
c) causing said vibratory transducer to generate a vibration motion in said vibratable element at said first imaging frequency;
d) causing said vibratable element to oscillate said target organ at said second imaging frequency;
e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected;
f) employing said ultrasound imaging system to receive said frequency signals, measure and process said Doppler shifts, and generate and display said images; and
g) using said images to visualize both said target organ and said distal end of said interventional medical device during an interventional procedure.

24. The method of claim 22 or 23, wherein said interventional medical device is an endovascular catheter and said endovascular catheter is introduced into a blood vessel lumen.

25. The method of claim 24, wherein said endovascular catheter further comprises a guide wire and intravascular ultra sound components.

26. The method of claim 24, wherein said vibratable element is a guide wire.

27. The method of claim 22 or 23, wherein said interventional medical device is an endolumenal catheter and said endolumenal catheter is introduced into a body lumen.

28. The method of claim 22 or 23, wherein said interventional medical device is a laparoscopic device.

29. The method of claim 22 or 23, wherein said interventional medical device is a trocar.

30. A method of rendering an interventional medical device and a blood vessel occlusion or stenosis detectable and treating said vessel occlusion or stenosis comprising:
a) introducing into a blood vessel having an occlusion or stenosis an apparatus comprising (i) an interventional medical device having a proximal end and a distal end and (ii) an imaging tool, said imaging tool comprising a vibratory transducer and a vibratable element, wherein said vibratable element is located at said distal end of said interventional medical device such that it is capable of contacting said vessel occlusion or stenosis, and wherein said vibratory transducer is capable of generating a plurality of vibration frequencies for vibrating said vibratable element at a first imaging frequency of about 2 Hz to about 2 kHz for Doppler imaging and at a therapeutic frequency of about 10 kHz to about 1 MHz sufficient for causing penetration or cavitation of vessel occlusions or clearing vessel stenosis;
b) advancing said distal end of said interventional medical device toward a proximal surface of said occlusion or stenosis;
c) causing said vibratory transducer to vibrate said vibratable element at said first imaging frequency;
d) advancing said interventional device until said vibratable element contacts said occlusion or stenosis, such that said vibratable element transmits a vibration to said occlusion or stenosis, causing said occlusion or stenosis to oscillate at said second imaging frequency;
e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected;
f) employing said ultrasound imaging system to receive said frequency signals, measure and process said Doppler shifts, and generate and display said images;
g) modulating said vibration frequency of said vibratory transducer to said therapeutic frequency and causing penetration or cavitation of said occlusion or at least partial ablation of said stenosis; and
h) alternately (i) locating a new proximal surface of said occlusion or stenosis and (ii) penetrating said occlusion or ablating said stenosis by modulating said vibratory transducer-generated vibration frequency between said first imaging frequency and said therapeutic frequency, until said occlusion is re-canalized or said stenosis is cleared.

31. A method of rendering an interventional medical device and a body lumen stenosis detectable and treating said body lumen stenosis comprising:
a) introducing into a body lumen having a stenosis an apparatus comprising (i) an interventional medical device having a proximal end and a distal end and (ii) an imaging tool, said imaging tool comprising a vibratory transducer and a vibratable element, wherein said vibratable element is located at said distal end of said interventional medical device such that it is capable of contacting said lumenal stenosis, and wherein said vibratory transducer is capable of generating a plurality of vibration frequencies for vibrating said vibratable element at a first imaging frequency of about 2 Hz to about 2 kHz for Doppler imaging and at a therapeutic frequency of about 10 kHz to about 1 MHz sufficient for clearing a lumenal stenosis;
b) advancing said distal end of said interventional medical device toward a proximal surface of said lumenal stenosis;
c) causing said vibratory transducer to vibrate said vibratable element at said first imaging frequency;
d) advancing said interventional device until said vibratable element contacts said lumenal stenosis, such that said vibratable element transmits a vibration to said lumenal stenosis, causing said lumenal stenosis to oscillate at a second imaging frequency;
e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected;
f) employing said ultrasound imaging system to receive said frequency signals, measure and process said Doppler shifts, and generate and display said images;
g) modulating said vibration frequency of said vibratory transducer to said therapeutic frequency and causing at least partial ablation of said lumenal stenosis; and
h) alternately (i) locating a new proximal surface of said lumenal stenosis and (ii) ablating said lumenal stenosis by modulating said vibratory transducer-generated vibration frequency between said first imaging frequency and said therapeutic frequency, until said lumenal stenosis is cleared.

32. A method of rendering an interventional medical device and a target organ in a patient detectable and treating said target organ comprising:
a) introducing into a patient's body near a target organ an apparatus comprising (i) an interventional medical device having a proximal end and a distal end and (ii) an imaging tool, said imaging tool comprising a vibratory transducer and a vibratable element, wherein said vibratory transducer is capable of generating a vibration motion in said vibratable element, said vibration motion having a first imaging vibration frequency of about 2 Hz to about 2 kHz for Doppler imaging, wherein said vibratable element is located at said distal end of said interventional medical device and is capable of causing said target organ to oscillate at a second imaging frequency of about 2 Hz to about 2 kHz, said target organ being in need of treatment, said interventional medical device being designed to treat said target organ;
b) advancing said distal end of said interventional medical device toward said target organ;
c) causing said vibratory transducer to vibrate said vibratable element at a first imaging vibration frequency;
d) advancing said interventional medical device until said vibrating vibratable element contacts said target organ, and causing said target organ to oscillate at a second imaging vibration frequency;
e) providing an ultrasound imaging system, comprising an ultrasound transducer for sending and receiving ultrasonic frequency signals, a processor for measuring and processing Doppler shifts in said ultrasonic frequency signals and for generating images of said target organ and said vibratable element and surrounding tissues, and an image screen for displaying said images, wherein said ultrasound transducer, said processor and said image screen are functionally connected;
f) employing said ultrasound imaging system to receive said frequency signals, measure and process said Doppler shifts, and generate and display said images;
g) using said images to visualize both said target organ and said distal end of said interventional medical device; and
h) treating said body organ with said interventional medical device.

33. The method of claim 32, wherein said interventional device is adapted for laparoscopic surgery.

34. The method of claim 32, wherein said interventional device is adapted for laparoscopic diagnostic applications.

35. The method of claim 32, wherein said interventional device is adapted for sampling tissues for biopsy and said target organ is an organ requiring a biopsy.

36. A system comprising:
an interventional medical device having a proximal end and a distal end,
an imaging tool comprising a vibratory transducer and a vibratable element, wherein said vibratory transducer is capable of generating at least one vibration frequency, including a first imaging frequency of about 2 Hz to about 2 kHz for generating a vibration motion in said vibratable element, and wherein said vibratable element is located at said distal end of said interventional medical device and is capable of causing a target organ to oscillate at a second imaging frequency of about 2 Hz to about 2 kHz with small amplitude;
an energy source functionally connected to said vibratory transducer;
a control unit functionally connected to said energy source, said control unit being capable of controlling and modulating said at least one vibration frequency generated by said vibratory transducer, via said energy source; and
an ultrasound imaging system for enabling an operator to localize said distal end of said interventional medical device and said target organ relative to surrounding tissues, said ultrasound imaging system comprising (i) an ultrasonic transducer for sending and receiving ultrasound wave signals, said ultrasonic transducer being capable of detecting Doppler shifts from movements of said vibrating vibratable element and said vibrating target organ, (ii) a processor and (iii) an image screen, wherein said ultrasonic transducer, said processor and said image screen are functionally connected.

37. The system of claim 36, wherein said interventional medical device is an endovascular catheter further comprising a guide wire and an intravascular ultra sound component.

38. The system of claim 36, wherein said interventional medical device is an endovascular catheter, wherein said target organ is a blood vessel occlusion or vascular stenosis, wherein said plurality of vibration frequencies includes a therapeutic frequency of about 10 kHz to about 1 MHz, and wherein said therapeutic frequency vibration is sufficient to enable said vibratable element to penetrate and traverse said vascular occlusion or to clear said vascular stenoses.

39. The system of claim 36, wherein said interventional medical device is an endolumenal catheter, wherein said target organ is a lumenal stenosis, wherein said plurality of vibration frequencies includes a therapeutic frequency of about 10 kHz to 1 MHz, and wherein said therapeutic frequency vibration is sufficient to enable said vibratable element to clear said lumenal stenosis.

40. The system of claim 38 or 39, wherein said vibratory transducer is selected from the group consisting of: a piezomotor, an electromechanical transducer, and a hydraulic energy source capable of generating hydraulic pressure waves or pulses.

41. The system of claim 38 or 39, wherein said vibratable element is selected from the group consisting of: a guide wire, a bellows, an elastic membrane, and a vibrating cap.
